(19) United States
(12) Patent Application Publication
(10) Pub. No.: US 2026/0172710 A1
(43) Pub. Date: Jun. 18, 2026

(54) DETECTION DEVICE

(71) Applicant: Japan Display Inc., Tokyo (JP)

(72) Inventors: Kaoru ITO, Tokyo (JP); Norio MAMBA, Tokyo (JP); Daichi ABE, Tokyo (JP); Yoshihiro SEKIGUCHI, Tokyo (JP)

(21) Appl. No.: 19/414,527

(22) Filed: Dec. 10, 2025

(30) Foreign Application Priority Data
Dec. 12, 2024 (JP) 2024-217767

(51) Int. Cl. H04N 25/77; H04N 25/709; H04N 25/78

(52) U.S. Cl. CPC H04N 25/77; H04N 25/709; H04N 25/78

(57) ABSTRACT

A detection device includes: a sensor panel having optical sensors each including a photodiode, a reset transistor configured to apply a reset potential to the photodiode, a source follower transistor configured to output a signal corresponding to a potential generated by the photodiode, and a readout transistor configured to read the signal from the source follower transistor; a light source; a detection circuit configured to acquire a potential corresponding to a bias current flowing through the readout transistor; and a control circuit configured to adjust a reset potential set value and/or a bias current set value such that an input potential acquired by the detection circuit via a signal transmission path between each optical sensor and the detection circuit falls within a predetermined range by driving the reset and readout transistors on while an object to be detected is not placed between the sensor panel and the light source.

### CROSS-REFERENCE TO RELATED APPLICATION

This application claims the benefit of priority from Japanese Patent Application No. 2024-217767 filed on Dec. 12, 2024, the entire contents of which are incorporated herein by reference.

### BACKGROUND

### 1. Technical Field

What is disclosed herein relates to a detection device.

### 2. Description of the Related Art

Japanese Patent Application Laid-open Publication No. H06-261737 (JP-A-H06-261737) discloses a biosensor that images, using a solid-state image sensing device, changes over time in state of samples to be cultured that are placed in a culture vessel together with a culture medium necessary for their growth. The samples to be cultured are bacteria, biological tissues such as cells, or the like.

In order to increase the accuracy of detection of the samples to be cultured when acquiring the changes over time in the samples to be cultured using a detection device, such as the biosensor described in JP-A-H06-261737 mentioned above, the biosensor-to-biosensor variations need to be absorbed to fall within a detection range of the sensor.

For the foregoing reasons, there is a need for a detection device capable of absorbing the device-to-device variations.

### SUMMARY

According to an aspect, a detection device includes: a plurality of optical sensors each including a photodiode, a reset transistor configured to apply a reset potential to a cathode of the photodiode, a source follower transistor configured to output a signal corresponding to a potential generated by the photodiode, and a readout transistor configured to read out an output signal of the source follower transistor; a sensor panel having a detection area in which the optical sensors are arranged in a planar configuration; a light source configured to emit light to an object to be detected placed between the light source and the sensor panel; a detection circuit configured to acquire a potential corresponding to a bias current that flows through the readout transistor; and a control circuit configured to control the sensor panel, the light source, and the detection circuit. The control circuit is configured to perform an initial setting process to adjust at least one of a set value of the reset potential or a set value of the bias current such that an input potential acquired by the detection circuit via a signal transmission path between each of the optical sensors and the detection circuit falls within a predetermined target setting range by controlling the reset transistor and the readout transistor to be on while the object to be detected is not placed between the sensor panel and the light source.

### BRIEF DESCRIPTION OF THE DRAWINGS

FIG. 1 is a diagram illustrating a main configuration of a detection device according to a first embodiment of the present disclosure;

FIG. 2 is a diagram illustrating a configuration example of a detection area and a wiring area;

FIG. 3 is a circuit diagram illustrating a circuit configuration of an optical sensor;

FIG. 4 is a schematic diagram illustrating a configuration example of a light-emitting element;

FIG. 5 is a schematic view illustrating a positional relation between main components of the detection device and an object to be detected, according to the first embodiment;

FIG. 6 is a cross-sectional view of the object to be detected in the schematic view illustrated in FIG. 5;

FIG. 7 is a first diagram for explaining an example of a detection operation in the detection device;

FIG. 8 is a second diagram for explaining the example of the detection operation in the detection device;

FIG. 9 is a flowchart illustrating an example of a scan process in the detection device;

FIG. 10 is a timing waveform diagram illustrating behavior of a reset transistor and a readout transistor in one frame period;

FIG. 11 is a diagram illustrating a configuration of a sensor panel and a detection circuit;

FIG. 12A is a schematic diagram schematically illustrating a distribution of an input potential of an analog front-end circuit (AFE) in a detection plane;

FIG. 12B is a schematic diagram schematically illustrating another distribution of the input potential of the AFE in the detection plane;

FIG. 12C is a schematic diagram schematically illustrating still another distribution of the input potential of the AFE in the detection plane;

FIG. 13 is a flowchart illustrating an example of an initial setting process in the detection device according to the first embodiment;

FIG. 14 is a sub-flowchart illustrating an example of a potential adjustment process;

FIG. 15 is a schematic plan view illustrating an example of the optical sensors that acquire an input potential VoRST of the AFE in the initial setting process;

FIG. 16 is a schematic diagram for explaining an operation in the potential adjustment process illustrated in FIG. 14;

FIG. 17 is a schematic diagram schematically illustrating a configuration example of a detection system including the detection device;

FIG. 18 is a schematic diagram illustrating a relation between one detection device and an external configuration in the detection system illustrated in FIG. 17;

FIG. 19 is a flowchart illustrating an example of the initial setting process in the detection device according to a second embodiment of the present disclosure; and

FIG. 20 is a sub-flowchart illustrating an example of a bias current adjustment process.

### DETAILED DESCRIPTION

The following describes embodiments of the present disclosure with reference to the drawings. What is disclosed herein is merely an example, and the present disclosure naturally encompasses appropriate modifications easily conceivable by those skilled in the art while maintaining the gist of the present invention. To further clarify the description, the drawings may schematically illustrate, for example, widths, thicknesses, and shapes of various parts as compared with actual aspects thereof. However, they are merely examples, and interpretation of the present disclosure is not limited thereto. The same element as that illustrated in a drawing that has already been discussed is denoted by the same reference numeral through the description and the drawings, and detailed description thereof may not be repeated where appropriate.

### First Embodiment

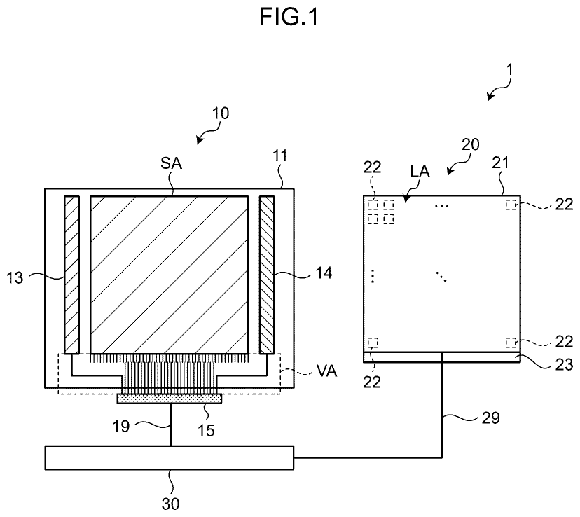

FIG. 1 is a diagram illustrating a main configuration of a detection device 1 according to a first embodiment of the present disclosure. The detection device 1 includes a sensor panel 10, a light source panel 20, and a control circuit 30. The sensor panel 10 and the light source panel 20 of the detection device 1 are coupled to the control circuit 30.

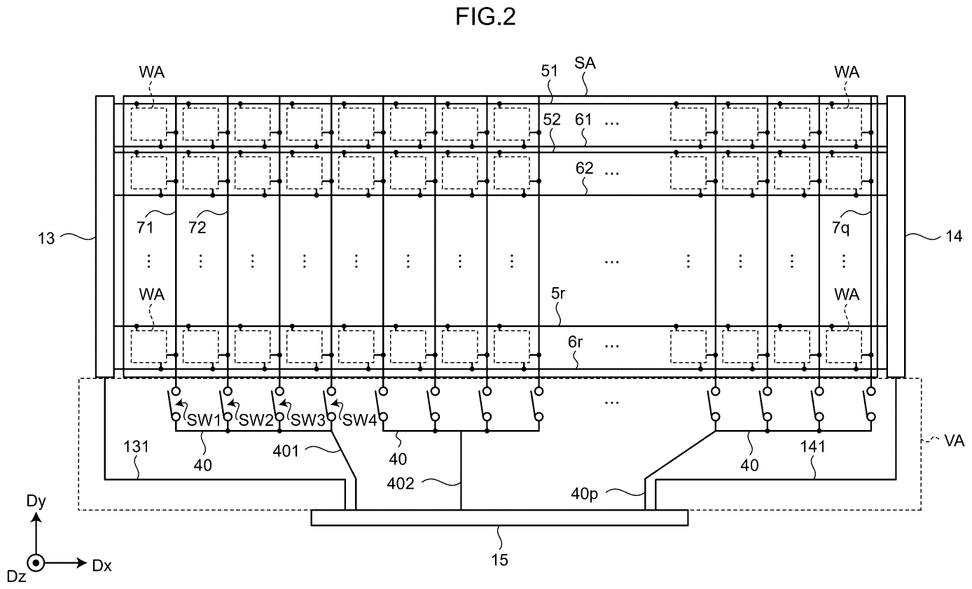

The sensor panel 10 is provided with a detection area SA (refer to FIG. 2) on a substrate 11. A reset circuit 13, a readout circuit 14, and a wiring area VA are provided on the substrate 11. Components on the detection area SA, the reset circuit 13, and the readout circuit 14 are coupled to a detection circuit 15 via the wiring area VA.

The light source panel 20 has a light-emitting area LA that evenly emits light to the detection area SA. As an exemplary aspect, the light source panel 20 is provided with a plurality of light-emitting elements on a substrate, and evenly emits light to the detection area SA using a diffuser plate 21, but the light source panel 20 is not limited to this aspect. A light-emitting element 22 is, for example, a light-emitting diode (LED), and is located in the light-emitting area LA. In the example illustrated in FIG. 1, a plurality of the light-emitting elements 22 are arranged in a matrix having a row-column configuration.

The light source panel 20 is provided with a light source drive circuit 23. Under the control of the control circuit 30, the light source drive circuit 23 controls whether to turn on each of the light-emitting elements 22 and the light intensity thereof when being turned on. The light-emitting elements 22 may be provided so as to be individually controllable in light emission, or may be provided so as to emit light collectively.

The control circuit 30 performs various types of control related to the operation of the detection device 1. Specifically, the control circuit 30 is a circuit, such as a field-programmable gate array (FPGA) or an application-specific integrated circuit (ASIC) that can implement a plurality of functions. The control circuit 30 is coupled to the detection circuit 15 via wiring 19 and obtains an output from the detection circuit 15. The control circuit 30 is coupled to the light source drive circuit 23 via wiring 29 and performs processing related to the lighting of the light-emitting elements 22, such as determination of lighting patterns of the light-emitting elements 22.

The control circuit 30 also performs processing related to detection of an object to be detected SUB (refer to FIG. 4) to be described later. This processing will be described later.

Although not illustrated in the drawings, the detection device 1 includes an analog-to-digital conversion circuit, a digital-to-analog conversion circuit, and other components. The analog-to-digital conversion circuit allows an output from an optical sensor WA (refer to FIG. 2) transmitted through the detection circuit 15 to be handled by arithmetic processing by the control circuit 30. The digital-to-analog conversion circuit makes digital signals generated by the arithmetic processing of the control circuit 30 usable for controlling operations of the sensor panel 10 and the light source panel 20. These circuits may be included, for example, in part or in whole in the control circuit 30, may be functions performed by circuits mounted on flexible printed circuits (FPCs) provided as the wiring 19 and the wiring 29, or may be implemented in other ways in the detection device 1.

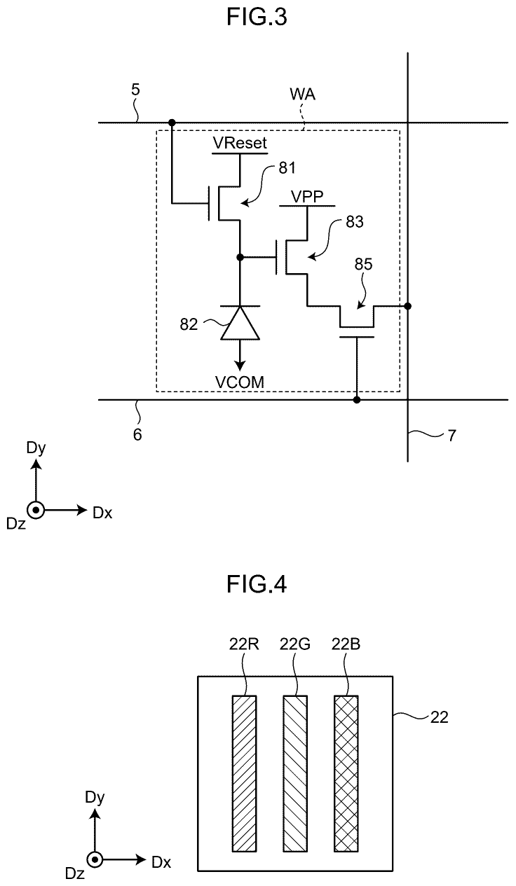

FIG. 2 is a diagram illustrating a configuration example of the detection area SA and the wiring area VA. A plurality of the optical sensors WA (FIG. 3) are provided in the detection area SA. Specifically, the example illustrated in FIG. 2 exemplifies an aspect in which the optical sensors WA are arranged in a matrix having a row-column configuration along a first direction Dx and a second direction Dy. The first direction Dx is orthogonal to the second direction Dy. In the following description, the term “third direction Dz” refers to a direction orthogonal to the first direction Dx and the second direction Dy.

The reset circuit 13 is coupled to reset control lines 51, 52, . . . , 5r. Hereinafter, the term “reset control line 5” refers to any one of the reset control lines 51, 52, . . . , 5r. The reset control line 5 is wiring along the first direction Dx. In the example illustrated in FIG. 2, r reset control lines 5 are arranged in the second direction Dy. r is a natural number equal to or larger than 2. The r reset control lines 5 are coupled, at first ends in the first direction Dx, to the reset circuit 13.

The readout circuit 14 is coupled to readout control lines 61, 62, . . . , 6r. Hereinafter, the term “readout control line 6” refers to any one of the readout control lines 61, 62, . . . , 6r. The readout control line 6 is wiring along the first direction Dx. In the example illustrated in FIG. 2, r readout control lines 6 are arranged in the second direction Dy. The r readout control lines 6 are coupled, at second ends in the first direction Dx, to the readout circuit 14.

As illustrated in FIG. 2, the reset control lines 5 and the readout control lines 6 are alternately arranged in the second direction Dy in the detection area SA. The reset circuit 13 and the readout circuit 14 illustrated in FIGS. 1 and 2 are arranged at locations facing each other with the detection area SA interposed therebetween, but the layout of the reset circuit 13 and the readout circuit 14 is not limited to this layout and can be changed as appropriate.

Signal lines 71, 72, . . . , 7q are also provided in the detection area SA. Hereinafter, the term “signal line 7” refers to any one of the signal lines 71, 72, . . . , 7q. The signal line 7 is wiring along the second direction Dy.

In the example illustrated in FIG. 2, q signal lines 7 are arranged in the first direction Dx. q is a natural number equal to or larger than 2. The q signal lines 7 are each coupled, at one end in the second direction Dy, to one of a plurality of switches (for example, switch SW1, SW2, SW3, or SW4) included in a multiplexer 40.

The multiplexer 40 is provided in the wiring area VA. The multiplexer 40 includes a plurality of switches. In the example illustrated in FIG. 2, the switches SW1, SW2, SW3, and SW4 are illustrated as the switches. The switches included in one multiplexer 40 are turned on (conducting state) at different times from one another. During a period when one of the switches included in the one multiplexer 40 is on (conducting state), the other switches are off (non-conducting state). The number of the multiplexers 40 depends on the number (q) of the signal lines 7. When the number of the switches is p, q/p is sufficient as the number of the multiplexers 40. When more than one multiplexers 40 are provided, each of the multiplexers 40 is coupled to the detection circuit 15 via an individual one of wiring lines 401, 402, . . . , 40p.

The coupling between the signal lines 7 and the detection circuit 15 via the multiplexer 40 is merely exemplary and is not limited to this example. The signal lines 7 may be individually directly coupled to the detection circuit 15 in the wiring area VA. In the wiring area VA, the reset circuit 13 is coupled to the detection circuit 15 via wiring 131. In the wiring area VA, the readout circuit 14 is coupled to the detection circuit 15 via wiring 141.

In detecting light using a photodiode 82 (refer to FIG. 3) provided in the optical sensor WA, the detection circuit 15 controls operation timing of the reset circuit 13 and the readout circuit 14. The detection circuit 15 receives the output from the optical sensor WA. The detection circuit 15 converts the signal received from the optical sensor WA into data that can be interpreted by the control circuit 30 and outputs the data to the control circuit 30. Hereafter, a detection value of each of the optical sensors WA output from the detection circuit 15 is also referred to as a “sensor value Raw”. The detection circuit 15 is, for example, a microcontroller unit (MCU) or a readout integrated circuit (ROIC) that includes an analog front-end circuit (AFE).

FIG. 3 is a circuit diagram illustrating a circuit configuration of the optical sensor WA. The first direction Dx and the second direction Dy in FIG. 3 merely correspond to the directions of the reset control line 5, the readout control line 6, and the signal line 7, and do not exactly indicate the relative positional relation of the circuit configuration in the optical sensor WA.

As illustrated in FIG. 3, a reset transistor 81, the photodiode 82, a source follower transistor 83, and a readout transistor 85 are provided in the optical sensor WA. In other words, the reset transistor 81, the source follower transistor 83, and the readout transistor 85 are provided correspondingly to one photodiode 82. The transistors included in the optical sensor WA are each configured as an n-type thin-film transistor (TFT). However, each of the transistors is not limited thereto, and may be configured as a p-type TFT.

A reference potential VCOM is applied to the anode of the photodiode 82. The cathode of the photodiode 82 is coupled to the gate of the source follower transistor 83 and one of the source and the drain of the reset transistor 81.

The gate of the reset transistor 81 is coupled to the reset control line 5. The other of the source and the drain of the reset transistor 81 is supplied with a reset potential VReset. When the reset transistor 81 is turned on (conducting state), the reset potential VReset is supplied to the cathode of the photodiode 82, and the potential of the cathode of the photodiode 82 is reset to the reset potential VReset. The reference potential VCOM is lower than the reset potential VReset. As a result, the photodiode 82 is driven into a reverse bias state.

The source follower transistor 83 is coupled between a terminal supplied with a source-of-output potential VPP and the readout transistor 85. The gate of the source follower transistor 83 is coupled to the cathode of the photodiode 82. The gate of the source follower transistor 83 is supplied with a voltage corresponding to a received light intensity of the photodiode 82. As a result, the source follower transistor 83 outputs a potential corresponding to the received light intensity of the photodiode 82 to the readout transistor 85.

The reset potential VReset, the reference potential VCOM, and the source-of-output potential VPP are supplied by the detection circuit 15 to the optical sensor WA based on, for example, electric power supplied via a power supply circuit (not illustrated) coupled to the detection circuit 15, but are not limited to being supplied in this way, and may be supplied in a different way as appropriate.

The readout transistor 85 is coupled between the source of the source follower transistor 83 and the signal line 7. The gate of the readout transistor 85 is coupled to the readout control line 6. When the readout transistor 85 is turned on (conducting state), the signal output from the source follower transistor 83, that is, the potential corresponding to the received light intensity of the photodiode 82 is output to the signal line 7.

In FIG. 3, the reset transistor 81 and the readout transistor 85 each have a single-gate structure. However, the reset transistor 81 and the readout transistor 85 may each have what is called a double-gate structure configured by coupling two transistors in series, or may have a configuration in which three or more transistors are coupled in series. The circuit of one optical sensor WA is not limited to the configuration including the three transistors of the reset transistor 81, the source follower transistor 83, and the readout transistor 85. The optical sensor WA may have a configuration including two transistors, or four or more transistors.

The reset circuit 13 is a circuit that drives the reset control lines 5 in the detection area SA. The reset circuit 13 includes a shift register circuit, for example.

In the present disclosure, the reset circuit 13 sequentially selects the reset control lines 5 based on various control signals such as start pulse signals and clock pulse signals supplied from the detection circuit 15, and supplies a reset control signal RST to the selected reset control lines 5. In other words, the reset circuit 13 simultaneously supplies the reset control signal RST to the optical sensors WA arranged in the first direction Dx, and sequentially supplies the reset control signal RST to the optical sensors WA arranged in the second direction Dy. This operation resets the potentials of the photodiodes 82 of the optical sensors WA coupled to the reset control lines 5 selected by the reset circuit 13 for the optical sensors WA.

The readout circuit 14 is a circuit that drives the readout control lines 6 in the detection area SA. The readout circuit 14 includes a shift register circuit, for example.

In the present disclosure, the readout circuit 14 sequentially selects the readout control lines 6 based on the various control signals such as the start pulse signals and the clock pulse signals supplied from the detection circuit 15, and supplies a readout control signal RD to the selected readout control lines 6. In other words, the readout circuit 14 simultaneously supplies the readout control signal RD to the optical sensors WA arranged in the first direction Dx, and sequentially supplies the readout control signal RD to the optical sensors WA arranged in the second direction Dy. As a result, the potentials of the optical sensors WA coupled to the readout control lines 6 selected by the readout circuit 14 are read out.

FIG. 4 is a schematic diagram illustrating a configuration example of the light-emitting element 22. As illustrated in FIG. 4, the light-emitting element 22 includes a first light-emitting element 22R, a second light-emitting element 22G, and a third light-emitting element 22B. The first light-emitting element 22R, the second light-emitting element 22G, and the third light-emitting element 22B emit light in different colors from one another. Specifically, the first light-emitting element 22R emits red (R) light, the second light-emitting element 22G emits green (G) light, and the third light-emitting element 22B emits blue (B) light. In this case, white light is emitted by simultaneously turning on the first light-emitting element 22R, the second light-emitting element 22G, and the third light-emitting element 22B.

FIG. 4 illustrates an exemplary configuration in which the longitudinal directions of the first light-emitting element 22R, the second light-emitting element 22G, and the third light-emitting element 22B extend along the second direction Dy, and the first light-emitting element 22R, the second light-emitting element 22G, and the third light-emitting element 22B are arranged in this order from one side to the other side in the first direction Dx. However, the shapes and positional relation of the first light-emitting element 22R, the second light-emitting element 22G, and the third light-emitting element 22B from a planar viewpoint are not limited to this exemplary configuration, and can be changed as appropriate. A single light-emitting element that emits white (W) light may be provided instead of the first light-emitting element 22R, the second light-emitting element 22G, and the third light-emitting element 22B.

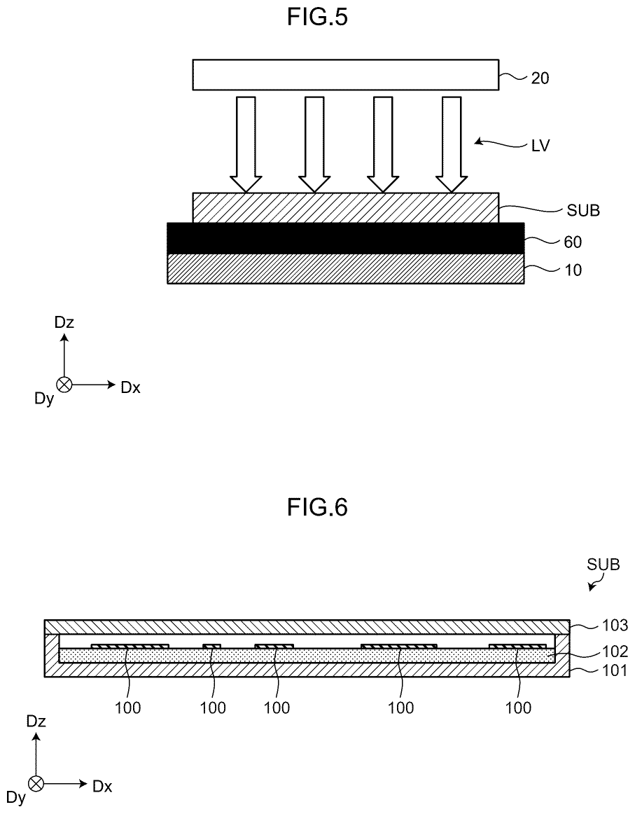

FIG. 5 is a schematic view illustrating a positional relation between main components of the detection device 1 and the object to be detected SUB, according to the first embodiment. FIG. 6 is a cross-sectional view of the object to be detected SUB in the schematic view illustrated in FIG. 5. In the detection device 1, the light source panel 20 and the sensor panel 10 are provided so as to face each other in the third direction Dz with the object to be detected SUB interposed therebetween.

As illustrated in FIG. 6, the object to be detected SUB is provided with a cover member 103 that is placed on the upper side of a light-transmitting placement substrate 101 formed of glass, for example, to cover a plurality of samples to be cultured 100. More specifically, the placement substrate 101 and the cover member 103 are a Petri dish, for example. The samples to be cultured 100 are placed on the placement substrate 101 together with a culture medium 102 (e.g., agar) and placed between the sensor panel 10 and the light source panel 20.

In the present disclosure, the samples to be cultured 100 are, for example, biological tissues such as bacteria or cells. A standard agar culture medium or a sheep blood agar culture medium exemplifies the culture medium 102 for culturing the samples to be cultured 100.

A light directivity control element 60 is provided between the object to be detected SUB and the sensor panel 10. The light directivity control element 60 is an optical element that transmits, toward the photodiode 82, components of the light emitted from the light source panel 20 that travel in a direction orthogonal to the sensor panel 10. The light directivity control element 60 is also called collimating apertures or a collimator. Alternatively, the light directivity control element 60 may be configured with a louver or microlenses instead of the collimator.

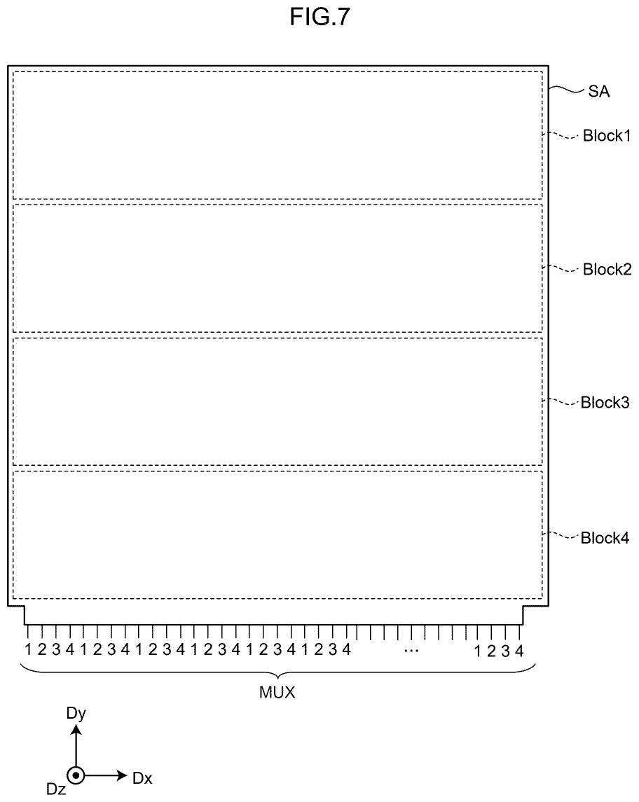

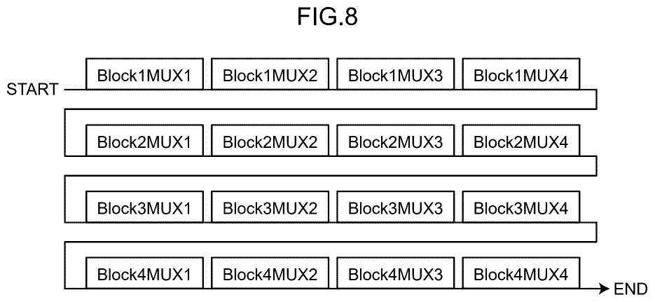

The following describes a specific example of a detection operation in one frame period in the detection device 1. FIG. 7 is a first diagram for explaining the example of the detection operation in the detection device 1. FIG. 8 is a second diagram for explaining the example of the detection operation in the detection device 1.

In an aspect illustrated in FIGS. 7 and 8, the detection area SA is divided into a plurality of segmented areas (blocks) in the second direction Dy. The example illustrated in FIG. 7 exemplifies the aspect in which the detection area SA is divided into four segmented areas Block1, Block2, Block3, and Block4. The number of the optical sensors WA arranged in the second direction Dy is preferably the same in each of the segmented areas Block1, Block2, Block3, and Block4.

In the aspect illustrated in FIGS. 7 and 8, MUX1, MUX2, MUX3, and MUX4 correspond to the switches SW1, SW2, SW3, and SW4 (refer to FIG. 2) included in each of the multiplexers 40.

In the detection device 1 according to such an aspect, the sensor values Raw are acquired in the following order: “Block1MUX1”, “Block1MUX2”, “Block1MUX3”, “Block1MUX4”, “Block2MUX1”, “Block2MUX2”, “Block2MUX3”, “Block2MUX4”, “Block3MUX1”, “Block3MUX2”, “Block3MUX3”, “Block3MUX4”, “Block4MUX1”, “Block4MUX2”, “Block4MUX3”, and “Block4MUX4”, as illustrated in FIG. 8.

In “Block1MUX1” illustrated in FIG. 8, the sensor values Raw of the optical sensors WA coupled to the detection circuit 15 via the switch SW1 of each of the multiplexers 40 in the segmented area Block1 are sequentially acquired while the switch SW1 of the multiplexer 40 is controlled to be on (conducting state).

In “Block1MUX2” illustrated in FIG. 8, the sensor values Raw of the optical sensors WA coupled to the detection circuit 15 via the switch SW2 of each of the multiplexers 40 in the segmented area Block1 are sequentially acquired while the switch SW2 of the multiplexer 40 is controlled to be on (conducting state).

In “Block1MUX3” illustrated in FIG. 8, the sensor values Raw of the optical sensors WA coupled to the detection circuit 15 via the switch SW3 of each of the multiplexers 40 in the segmented area Block1 are sequentially acquired while the switch SW3 of the multiplexer 40 is controlled to be on (conducting state).

In “Block1MUX4” illustrated in FIG. 8, the sensor values Raw of the optical sensors WA coupled to the detection circuit 15 via the switch SW4 of each of the multiplexers 40 in the segmented area Block1 are sequentially acquired while the switch SW4 of the multiplexer 40 is controlled to be on (conducting state).

In “Block2MUX1” illustrated in FIG. 8, the sensor values Raw of the optical sensors WA coupled to the detection circuit 15 via the switch SW1 of each of the multiplexers 40 in the segmented area Block2 are sequentially acquired while the switch SW1 of the multiplexer 40 is controlled to be on (conducting state).

In “Block2MUX2” illustrated in FIG. 8, the sensor values Raw of the optical sensors WA coupled to the detection circuit 15 via the switch SW2 of each of the multiplexers 40 in the segmented area Block2 are sequentially acquired while the switch SW2 of the multiplexer 40 is controlled to be on (conducting state).

In “Block2MUX3” illustrated in FIG. 8, the sensor values Raw of the optical sensors WA coupled to the detection circuit 15 via the switch SW3 of each of the multiplexers 40 in the segmented area Block2 are sequentially acquired while the switch SW3 of the multiplexer 40 is controlled to be on (conducting state).

In “Block2MUX4” illustrated in FIG. 8, the sensor values Raw of the optical sensors WA coupled to the detection circuit 15 via the switch SW4 of each of the multiplexers 40 in the segmented area Block2 are sequentially acquired while the switch SW4 of the multiplexer 40 is controlled to be on (conducting state).

In “Block3MUX1” illustrated in FIG. 8, the sensor values Raw of the optical sensors WA coupled to the detection circuit 15 via the switch SW1 of each of the multiplexers 40 in the segmented area Block3 are sequentially acquired while the switch SW1 of the multiplexer 40 is controlled to be on (conducting state).

In “Block3MUX2” illustrated in FIG. 8, the sensor values Raw of the optical sensors WA coupled to the detection circuit 15 via the switch SW2 of each of the multiplexers 40 in the segmented area Block3 are sequentially acquired while the switch SW2 of the multiplexer 40 is controlled to be on (conducting state).

In “Block3MUX3” illustrated in FIG. 8, the sensor values Raw of the optical sensors WA coupled to the detection circuit 15 via the switch SW3 of each of the multiplexers 40 in the segmented area Block3 are sequentially acquired while the switch SW3 of the multiplexer 40 is controlled to be on (conducting state).

In “Block3MUX4” illustrated in FIG. 8, the sensor values Raw of the optical sensors WA coupled to the detection circuit 15 via the switch SW4 of each of the multiplexers 40 in the segmented area Block3 are sequentially acquired while the switch SW4 of the multiplexer 40 is controlled to be on (conducting state).

In “Block4MUX1” illustrated in FIG. 8, the sensor values Raw of the optical sensors WA coupled to the detection circuit 15 via the switch SW1 of each of the multiplexers 40 in the segmented area Block4 are sequentially acquired while the switch SW1 of the multiplexer 40 is controlled to be on (conducting state).

In “Block4MUX2” illustrated in FIG. 8, the sensor values Raw of the optical sensors WA coupled to the detection circuit 15 via the switch SW2 of each of the multiplexers 40 in the segmented area Block4 are sequentially acquired while the switch SW2 of the multiplexer 40 is controlled to be on (conducting state).

In “Block4MUX3” illustrated in FIG. 8, the sensor values Raw of the optical sensors WA coupled to the detection circuit 15 via the switch SW3 of each of the multiplexers 40 in the segmented area Block4 are sequentially acquired while the switch SW3 of the multiplexer 40 is controlled to be on (conducting state).

In “Block4MUX4” illustrated in FIG. 8, the sensor values Raw of the optical sensors WA coupled to the detection circuit 15 via the switch SW4 of each of the multiplexers 40 in the segmented area Block4 are sequentially acquired while the switch SW4 of the multiplexer 40 is controlled to be on (conducting state).

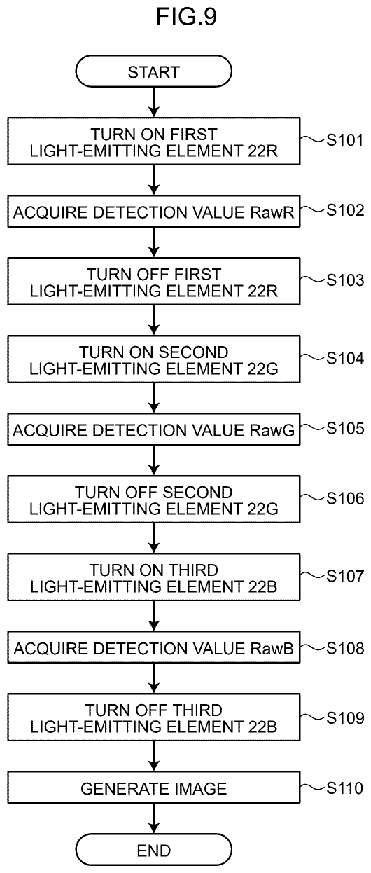

FIG. 9 is a flowchart illustrating an example of a scan process in the detection device 1. In the present disclosure, the term “scan process” refers to a process to generate an image of the object to be detected SUB by turning on the light source panel 20 to emit the light to the sensor panel 10 and acquiring the sensor value of each of the optical sensors WA corresponding to the amount of light received by the photodiode 82 included in the optical sensor WA.

In the scan process with reference to FIG. 9, the control circuit 30 first turns on the first light-emitting elements 22R (Step S101), acquires a sensor value RawR of each of the optical sensors WA (Step S102), and turns off the first light-emitting elements 22R (Step S103).

The control circuit 30 then turns on the second light-emitting elements 22G (Step S104), acquires a sensor value RawG of each of the optical sensors WA (Step S105), and turns off the second light-emitting elements 22G (Step S106).

The control circuit 30 then turns on the third light-emitting elements 22B (Step S107), acquires a sensor value RawB for each of the optical sensors WA (Step S108), and turns off the third light-emitting elements 22B (Step S109).

Then, the control circuit 30 generates the image of the object to be detected SUB in the plane of the detection area SA by combining the acquired sensor values RawR, RawG, and RawB of the respective optical sensors WA (Step S110).

By performing the scan process described above at intervals of a predetermined wait time, changes over time of the state of the samples to be cultured 100 can be acquired. The wait time in the present disclosure is five minutes, for example.

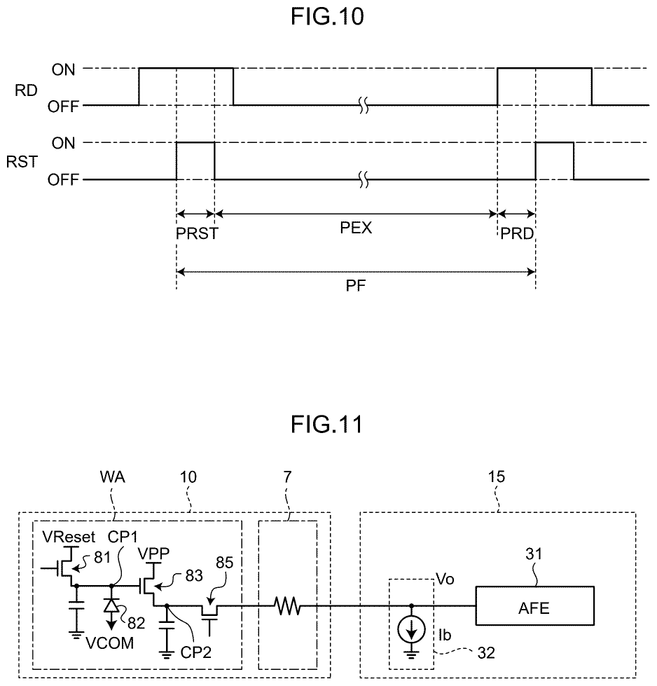

FIG. 10 is a timing waveform diagram illustrating behavior of the reset transistor 81 and the readout transistor 85 in one frame period PF. The following describes circuit operations in a reset period PRST, an exposure period PEX, and a readout period PRD with reference to FIG. 10. In FIG. 10, RST indicates a reset signal supplied from the reset circuit 13, and RD indicates a readout signal supplied from the readout circuit 14. FIG. 11 is a diagram illustrating a configuration of the sensor panel 10 and the detection circuit 15.

In the present disclosure, the detection circuit 15 includes an analog front end (AFE) 31. The detection circuit 15 also includes a constant-current source 32 for applying a bias current Ib to the readout transistor 85 via the signal line 7.

When the reset transistor 81 is turned on (conducting state) in the reset period PRST, the potential of a cathode CP1 of the photodiode 82 is initialized to the reset potential VReset.

In the subsequent exposure period PEX, an electric charge, which has been stored in (parasitic capacitance of) the cathode of the photodiode 82 according to the intensity of the light detected by the photodiode 82, is discharged and the potential of the cathode CP1 of the photodiode 82 decreases. As a result, the potential of a coupling point CP2 between the source follower transistor 83 and the readout transistor 85 decreases.

The potential of the coupling point CP2 between the source follower transistor 83 and the readout transistor 85 is lower than that of the cathode CP1 of the photodiode 82 by a voltage (threshold voltage Vth) between the gate and the source of the source follower transistor 83. The threshold voltage Vth of each of the transistors included in the optical sensor WA varies from sensor panel 10 to sensor panel 10.

When the readout transistor 85 is turned on (conducting state) in the readout period PRD after the exposure period PEX, an electric charge, which has been stored in (parasitic capacitance of) the coupling point CP2 between the source follower transistor 83 and the readout transistor 85 during a period when the readout transistor 85 has been controlled to be off, is supplied to the detection circuit 15 via (wiring resistance of) the signal line 7, and a potential Vo corresponding to the bias current Ib is received by the AFE 31. Hereinafter, the potential Vo received by the AFE 31 is also referred to as an “input potential Vo of the AFE 31”.

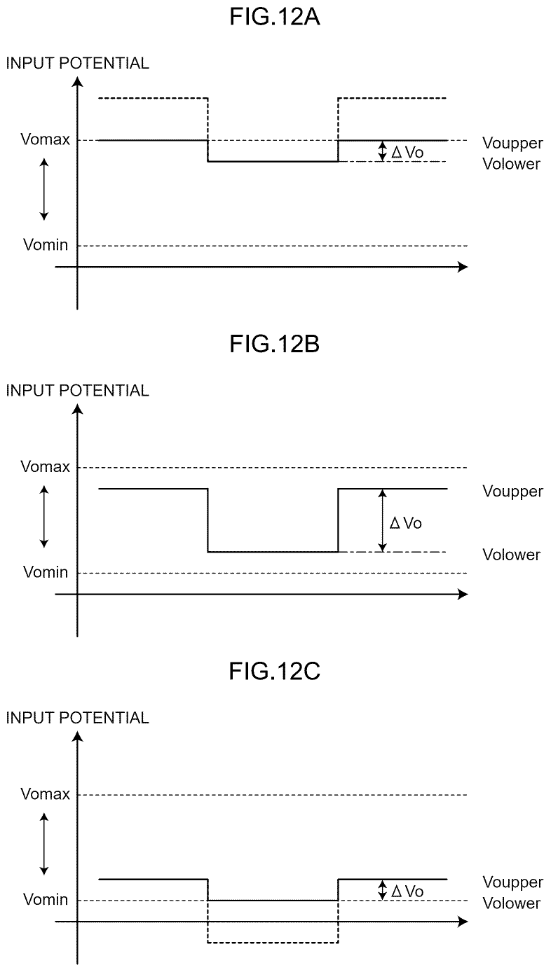

FIGS. 12A, 12B, and 12C are schematic diagrams schematically illustrating distributions of the input potential Vo of the AFE 31 in a detection plane. FIGS. 12A, 12B, and 12C illustrate the distributions of the input potential Vo of the AFE 31 while one sample to be cultured 100 is present in the center of the culture medium 102.

Vomin illustrated in FIGS. 12A, 12B, and 12C represents the lower limit value of the input potential Vo of the AFE 31 (hereinafter, also referred to as a “lower limit input potential Vomin of the AFE 31”) in the detection circuit 15. Vomax illustrated in FIGS. 12A, 12B, and 12C represents the upper limit value of the input potential Vo of the AFE 31 (hereinafter, also referred to as an “upper limit input potential Vomax of the AFE 31”) in the detection circuit 15. In the present disclosure, a range from the lower limit input potential Vomin to the upper limit input potential Vomax of the AFE 31 is defined as being within a detection range of the input potential Vo of the AFE 31 in the detection circuit 15.

The example illustrated in FIG. 12B illustrates an aspect in which an input potential Voupper of the AFE 31 in an area corresponding to the culture medium 102 and an input potential Volower of the AFE 31 in an area corresponding to the sample to be cultured 100 fall within the detection range of the input potential Vo of the AFE 31 in the detection circuit 15. More specifically, in the example illustrated in FIG. 12B, the input potential Volower of the AFE 31 in the area corresponding to the sample to be cultured 100 is equal to or higher than the lower limit input potential Vomin of the AFE 31 (Volower≥Vomin), and the input potential Voupper of the AFE 31 in the area corresponding to the culture medium 102 is equal to or lower than the upper limit input potential Vomax of the AFE 31 (Voupper≤Vomax).

In contrast, if the threshold voltage Vth of the source follower transistor 83 included in the optical sensor WA is relatively lower, the input potential Voupper of the AFE 31 in the area corresponding to the culture medium 102 may be limited to the upper limit input potential Vomax of the AFE 31, as illustrated in FIG. 12A. Consequently, the difference ΔVo between the input potential Voupper of the AFE 31 in the area corresponding to the culture medium 102 and the input potential Volower of the AFE 31 in the area corresponding to the sample to be cultured 100 may become smaller.

For example, if the threshold voltage Vth of the source follower transistor 83 is relatively higher, the input potential Volower of the AFE 31 in the area corresponding to the sample to be cultured 100 may be limited to the lower limit input potential Vomin of the AFE 31, as illustrated in FIG. 12C. Consequently, the difference ΔVo between the input potential Volower of the AFE 31 in the area corresponding to the sample to be cultured 100 and the input potential Voupper of the AFE 31 in the area corresponding to the culture medium 102 may become smaller.

Thus, when the threshold voltage Vth of each of the transistors included in the optical sensor WA varies from sensor panel 10 to sensor panel 10, changes over time of the state of the sample to be cultured may not be acquired properly.

In the first embodiment, the reset potential VReset and the reference potential VCOM supplied to the optical sensor WA are adjusted in an initial setting process before the scan process starts, and, the subsequent scan process is performed by applying the reset potential VReset and the reference potential VCOM that have been set in the initial setting process. The following describes a specific example of the initial setting process according to the first embodiment.

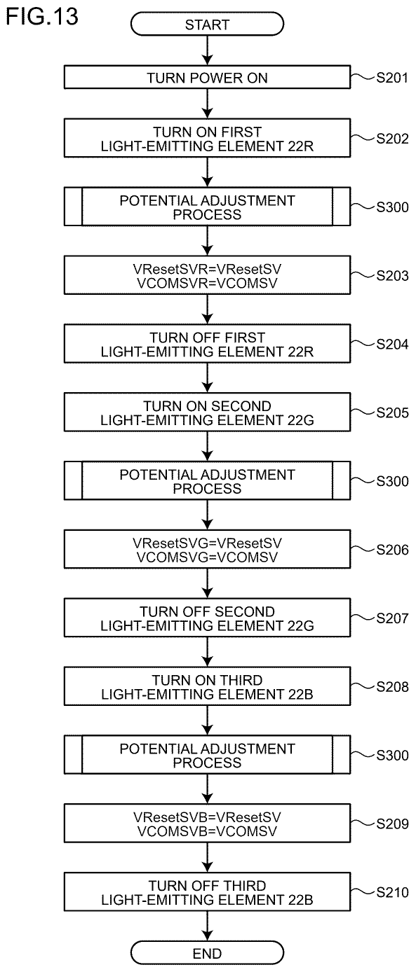

FIG. 13 is a flowchart illustrating an example of the initial setting process in the detection device 1 according to the first embodiment. The initial setting process illustrated in FIG. 13 is performed while the object to be detected SUB is not provided between the sensor panel 10 and the light source panel 20 before the start of the scan process illustrated in FIG. 9.

When the power of the detection device 1 is turned on (Step S201), the control circuit 30 first turns on the first light-emitting elements 22R (Step S202), and performs a potential adjustment process to set a reset potential VResetSVR and a reference potential VCOMSVR to be applied when the first light-emitting elements 22R are turned on in the scan process illustrated in FIG. 9 (Step S300).

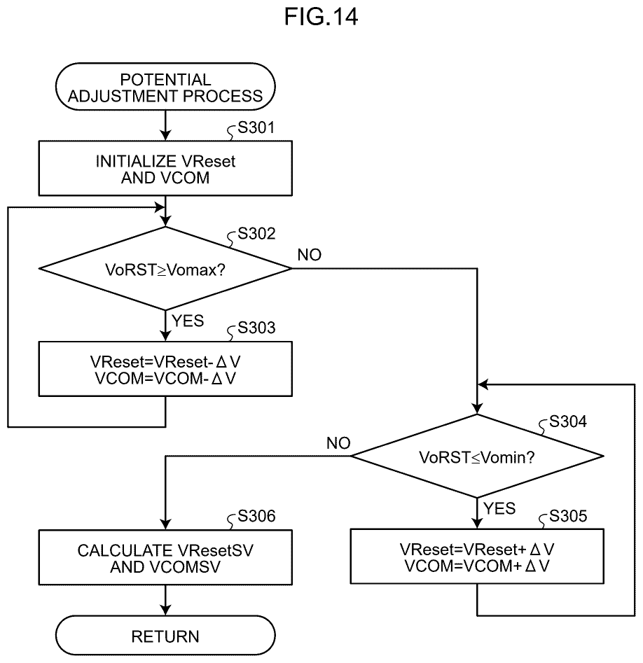

FIG. 14 is a sub-flowchart illustrating an example of the potential adjustment process. In the potential adjustment process illustrated in FIG. 14, the control circuit 30 first initializes the reset potential VReset and the reference potential VCOM that are supplied to each of the optical sensors WA (Step S301). Initial values of the reset potential VReset and the reference potential VCOM supplied to the optical sensor WA in the potential adjustment process illustrated in FIG. 14 have been preset.

The control circuit 30 turns on (conducting state) both the reset transistor 81 and the readout transistor 85 to acquire the input potential VoRST of the AFE 31, and determines whether the acquired input potential VoRST of the AFE 31 is equal to or higher than the upper limit input potential Vomax of the AFE 31 (Step S302).

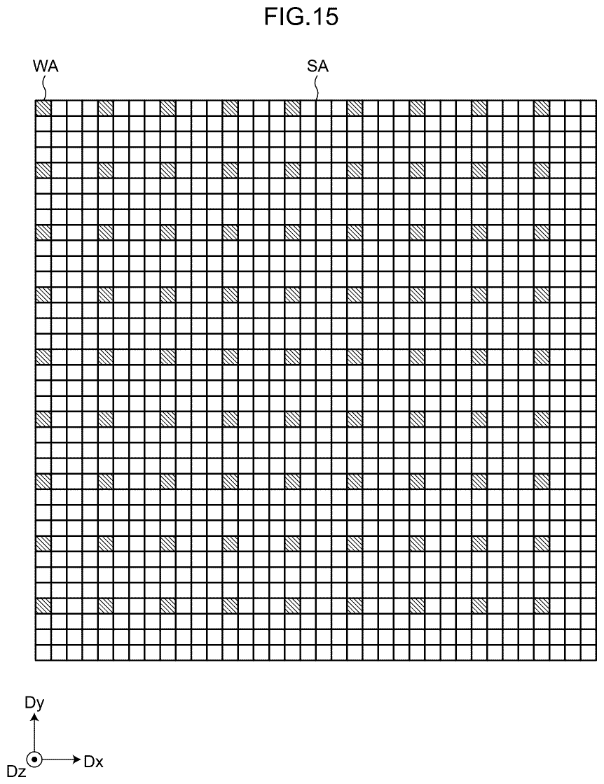

The input potential VoRST acquired in the initial setting process may be, for example, the average value of the input potentials VoRST acquired from the optical sensors WA in the detection area SA, as illustrated in FIG. 15.

FIG. 15 is a schematic plan view illustrating an example of the optical sensors WA that acquire the input potential VoRST of the AFE 31 in the initial setting process. In the example illustrated in FIG. 15, the optical sensors WA that acquire the input potential VoRST of the AFE 31 are indicated by hatching.

FIG. 15 illustrates an aspect in which the input potential VoRST of the AFE 31 is acquired from the optical sensors WA apart from one another by predetermined distances in the first direction Dx and the second direction Dy in the detection area SA by intermittently driving the optical sensors WA arranged in the first direction Dx and the optical sensors WA arranged in the second direction Dy.

As an alternative aspect, the average value of the input potentials VoRST of the AFE 31 acquired from the optical sensors WA in the segmented areas Block2 and Block3 illustrated in FIG. 7 may be used.

Referring back to FIG. 14, if the input potential VoRST of the AFE 31 is equal to or higher than the upper limit input potential Vomax of the AFE 31 (VoRST≥Vomax; Yes at Step S302), the control circuit 30 subtracts ΔV from both the reset potential VReset and the reference potential VCOM (VReset=VReset−ΔV, and VCOM=VCOM−ΔV; Step S303) to obtain the input potential VoRST of the AFE 31, and repeatedly performs the processes at Steps S302 and S303 (first process). ΔV that is subtracted from the reset potential VReset and the reference potential VCOM at Step S303 is 0.1 V, for example.

If the input potential VoRST of the AFE 31 is lower than the upper limit input potential Vomax of the AFE 31 (VoRST<Vomax; No at Step S302), the control circuit 30 then determines whether the input potential VoRST of the AFE 31 is equal to or lower than the lower limit input potential Vomin of the AFE 31 (Step S304).

If the input potential VoRST of the AFE 31 is equal to or lower than the lower limit input potential Vomin of the AFE 31 (VoRST≤Vomin; Yes at Step S304), the control circuit 30 adds ΔV to both the reset potential VReset and the reference potential VCOM (VReset=VReset+ΔV, and VCOM=VCOM+ΔV; Step S305) to obtain the input potential VoRST of the AFE 31, and repeatedly performs the processes at Steps S304 and S305 (second process). ΔV that is added to the reset potential VReset and the reference potential VCOM at Step S305 is 0.1 V, for example.

If the input potential VoRST of the AFE 31 exceeds the lower limit input potential Vomin of the AFE 31 (VoRST>Vomin; No at Step S304), the control circuit 30 uses Expressions (1) and (2) given below to calculate a reset potential set value VResetSV and a reference potential set value VCOMSV that are to be applied in the scan process illustrated in FIG. 9 (Step S306) (third process).

$$V ResetS V = V Reset + ( V otarget - V oRST ) \qquad ( 1 )$$

$$V COMS V = V COM + ( V otarget - V oRST ) \qquad ( 2 )$$

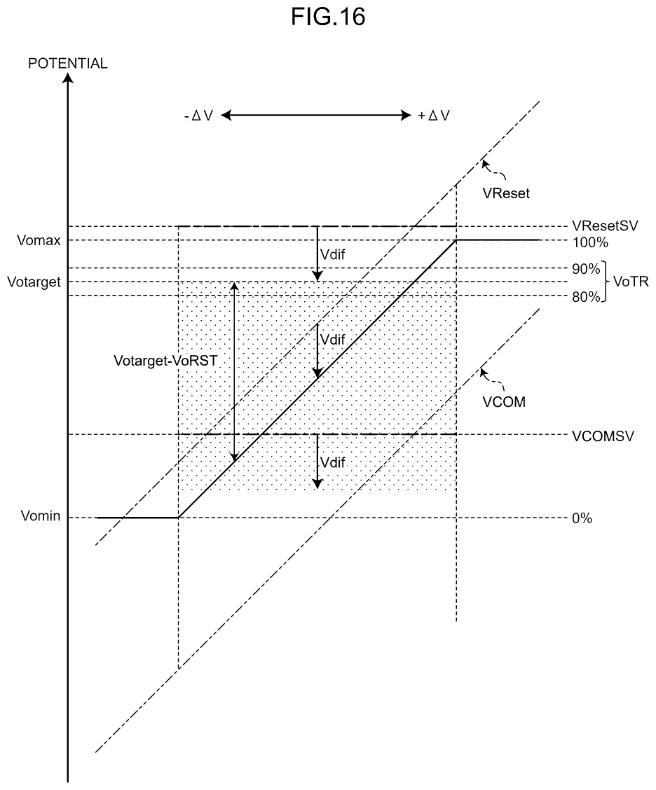

Votarget in Expressions (1) and (2) given above represents a target value of the input potential VoRST of the AFE 31 (hereinafter, also referred to as an “input potential target value Votarget of the AFE 31”). The input potential target value Votarget of the AFE 31 has been preset within a target setting range VoTR of 80% to 90% with respect to the upper limit input potential Vomax of the AFE 31 (refer to FIG. 16).

The following describes a specific example of an operation in the potential adjustment process illustrated in FIG. 14. FIG. 16 is a schematic diagram for explaining the operation in the potential adjustment process illustrated in FIG. 14.

As described above, if the input potential VoRST of the AFE 31 is equal to or higher than the upper limit input potential Vomax of the AFE 31 (VoRST≥Vomax; Yes at Step S302), the control circuit 30 subtracts ΔV from both the reset potential VReset and the reference potential VCOM (VReset=VReset−ΔV, and VCOM=VCOM−ΔV; Step S303) (first process) until the input potential VoRST of the AFE 31 becomes lower than the upper limit input potential Vomax of the AFE 31 (VoRST<Vomax; No at Step S302).

If the input potential VoRST of the AFE 31 is equal to or lower than the lower limit input potential Vomin of the AFE 31 (VoRST≤Vomin; Yes at Step S304), the control circuit 30 adds ΔV to both the reset potential VReset and the reference potential VCOM (VReset=VReset+ΔV, and VCOM=VCOM+ΔV; Step S305) (second process) until the input potential VoRST of the AFE 31 exceeds the lower limit input potential Vomin of the AFE 31 (VoRST>Vomin; No at Step S304).

Then, as illustrated in FIG. 16, if the input potential VoRST of the AFE 31 exceeds the lower limit input potential Vomin of the AFE 31 (VoRST>Vomin; No at Step S304) and the input potential VoRST of the AFE 31 is lower than the upper limit input potential Vomax of the AFE 31 (VoRST<Vomax; No at Step S302), the control circuit 30 uses Expressions (1) and (2) given above to calculate the reset potential set value VResetSV and the reference potential set value VCOMSV to be applied in the scan process illustrated in FIG. 9 (Step S306) (third process).

At this time, the control circuit 30 sets the reset potential set value VResetSV and the reference potential set value VCOMSV such that the potential difference between the ends of the photodiode 82 is constant. More specifically, the control circuit 30 adds the difference between the input potential target value Votarget of the AFE 31 and the input potential VoRST of the AFE 31 (Votarget−VoRST) to the reset potential VReset to calculate the reset potential set value VResetSV to be applied in the scan process illustrated in FIG. 9. The control circuit 30 also adds the difference between the input potential target value Votarget of the AFE 31 and the input potential VoRST of the AFE 31 (Votarget−VoRST) to the reference potential VCOM to calculate the reference potential set value VCOMSV to be applied in the scan process illustrated in FIG. 9. As a result, the sensitivity of detecting the object to be detected SUB is kept constant.

The input potential VoRST of the AFE 31 acquired in the potential adjustment process illustrated in FIG. 14 described above is a potential lower than the reset potential VReset by a potential Vdif. The potential Vdif is a potential obtained by summing the threshold voltage Vth of the source follower transistor 83 and a potential drop corresponding to a resistance component of a signal transmission path between the optical sensor WA and the detection circuit 15 and the bias current Ib. Therefore, in the scan process illustrated in FIG. 9, the input potential Vo of the AFE 31 can be a potential within a range from a potential lower than the reference potential set value VCOMSV by the potential Vdif to a potential lower than the reset potential set value VResetSV by the potential Vdif, as indicated by hatching in FIG. 16.

Referring back to the initial setting process illustrated in FIG. 13, the control circuit 30 sets the reset potential set value VResetSV and the reference potential set value VCOMSV, both calculated at Step S306, as the reset potential VResetSVR and the reference potential VCOMSVR when the first light-emitting elements 22R are turned on during the scan process illustrated in FIG. 9 (Step S203).

The control circuit 30 then turns off the first light-emitting elements 22R (Step S204), turns on the second light-emitting elements 22G (Step S205), and performs the potential adjustment process to set a reset potential VResetSVG and a reference potential VCOMSVG that are to be applied when the second light-emitting elements 22G are turned on in the scan process illustrated in FIG. 9 (Step S300). The potential adjustment process to set the reset potential VResetSVG and the reference potential VCOMSVG is the same as the above-described potential adjustment process to set the reset potential VResetSVR and the reference potential VCOMSVR when the first light-emitting elements 22R are turned on, and therefore, will not be described in detail.

Referring back to the initial setting process illustrated in FIG. 13, the control circuit 30 sets the reset potential set value VResetSV and the reference potential set value VCOMSV, both calculated at Step S306, as the reset potential VResetSVG and the reference potential VCOMSVG when the second light-emitting elements 22G are turned on in the scan process illustrated in FIG. 9 (Step S206).

The control circuit 30 then turns off the second light-emitting elements 22G (Step S207), turns on the third light-emitting elements 22B (Step S208), and performs the potential adjustment process to set a reset potential VResetSVB and a reference potential VCOMSVB that are to be applied when the third light-emitting elements 22B are turned on in the scan process illustrated in FIG. 9 (Step S300). The potential adjustment process to set the reset potential VResetSVB and the reference potential VCOMSVB is the same as: the above-described potential adjustment process to set the reset potential VResetSVR and the reference potential VCOMSVR when the first light-emitting elements 22R are turned on; and the above-described potential adjustment process to set the reset potential VResetSVG and the reference potential VCOMSVG when the second light-emitting elements 22G are turned on. The potential adjustment process to set the reset potential VResetSVB and the reference potential VCOMSVB, therefore, will not be described in detail.

Referring back to the initial setting process illustrated in FIG. 13, the control circuit 30 sets the reset potential set value VResetSV and the reference potential set value VCOMSV, both calculated at Step S306, as the reset potential VResetSVB and the reference potential VCOMSVB when the third light-emitting elements 22B are turned on in the scan process illustrated in FIG. 9 (Step S209).

The control circuit 30 then turns off the third light-emitting elements 22B (Step S210) and ends the initial setting process illustrated in FIG. 13.

When performing the scan process illustrated in FIG. 9, the detection device 1 according to the first embodiment applies the reset potential set value VResetSV and the reference potential set value VCOMSV that have been set after the initial setting process illustrated in FIG. 13. As a result, the changes over time of the state of the samples to be cultured 100 can fall within the detection range in the detection circuit 15 regardless of sensor-panel-to-sensor-panel variation in the threshold voltage Vth of each of the transistors included in the optical sensor WA.

As described above, in the initial setting process before starting the scan process, the detection device 1 according to the first embodiment adjusts the reset potential VReset and the reference potential VCOM to be supplied to the optical sensor WA; and, when performing the subsequent scan process, the detection device 1 applies the reset potential set value VResetSV and the reference potential set value VCOMSV that have been set in the initial setting process. As a result, the changes over time of the state of the samples to be cultured 100 can be acquired properly regardless of sensor-panel-to-sensor-panel variation in the threshold voltage Vth of each of the transistors included in the optical sensor WA.

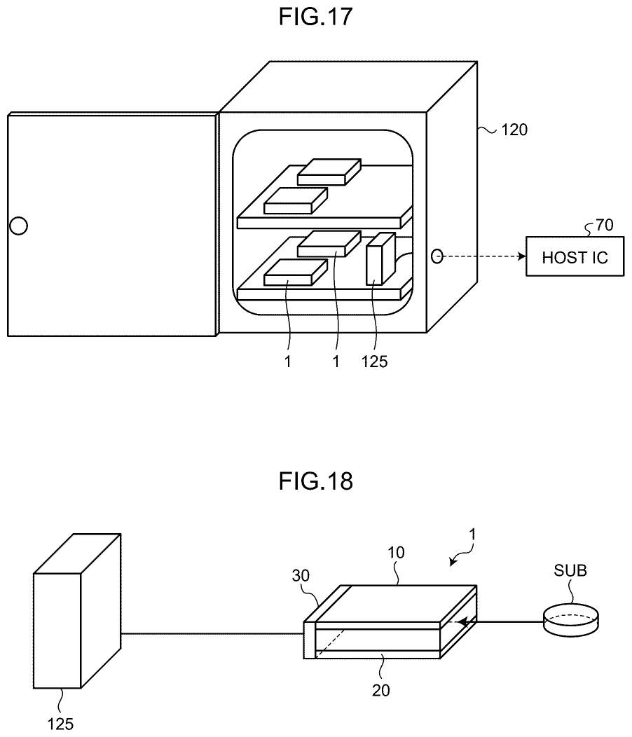

FIG. 17 is a schematic diagram schematically illustrating a configuration example of a detection system including the detection device 1. In the detection system illustrated in FIG. 17, a plurality of the detection devices 1 are electrically coupled to a common host integrated circuit (IC) 70 via a coupling circuit 125.

An incubator 120 is maintained such that an environment (temperature, humidity, and the like) therein is suitable for culturing the object to be detected while a door is closed. In the detection system illustrated in FIG. 17, the detection devices 1 are placed in the incubator 120.

FIG. 18 is a schematic diagram illustrating a relation between one detection device 1 and an external configuration in the detection system illustrated in FIG. 17. As illustrated in FIG. 18, the detection device 1 is coupled to the coupling circuit 125 by coupling the control circuit 30 to the coupling circuit 125. As illustrated in FIG. 18, the sensor panel 10 faces the light source panel 20. A gap where the object to be detected SUB can be placed is provided between the sensor panel 10 and the light source panel 20.

### Second Embodiment

In a second embodiment of the present disclosure, the bias current Ib to flow through the signal transmission path between the optical sensor WA and the detection circuit 15 is adjusted in the initial setting process before starting the scan process, and the bias current Ib set in the initial setting process is applied to perform the subsequent scan process. The following describes a specific example of the initial setting process according to the second embodiment.

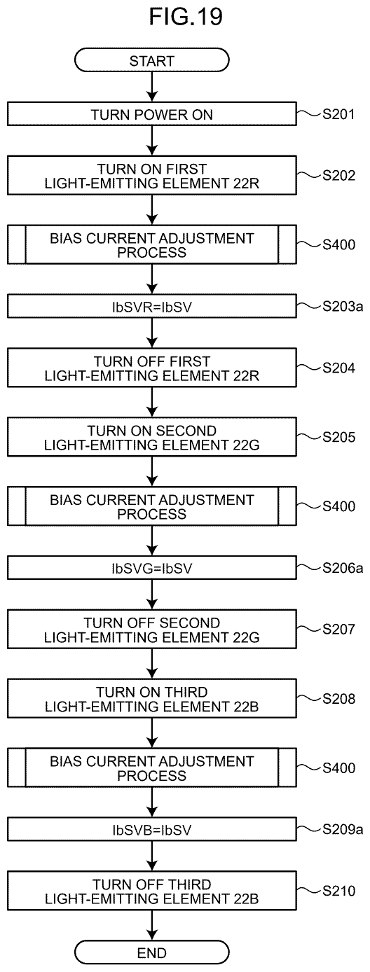

FIG. 19 is a flowchart illustrating an example of the initial setting process in the detection device according to the second embodiment. In the same way as in the first embodiment, the initial setting process illustrated in FIG. 19 is performed while the object to be detected SUB is not provided between the sensor panel 10 and the light source panel 20 before the start of the scan process illustrated in FIG. 9. The configuration of the detection device 1 according to the second embodiment is the same as that according to the first embodiment, and therefore, will not be described in detail.

When the power of the detection device 1 is turned on (Step S201), the control circuit 30 first turns on the first light-emitting elements 22R (Step S202), and performs a bias current adjustment process to set the bias current Ib to be applied when the first light-emitting elements 22R are turned on in the scan process illustrated in FIG. 9 (Step S400).

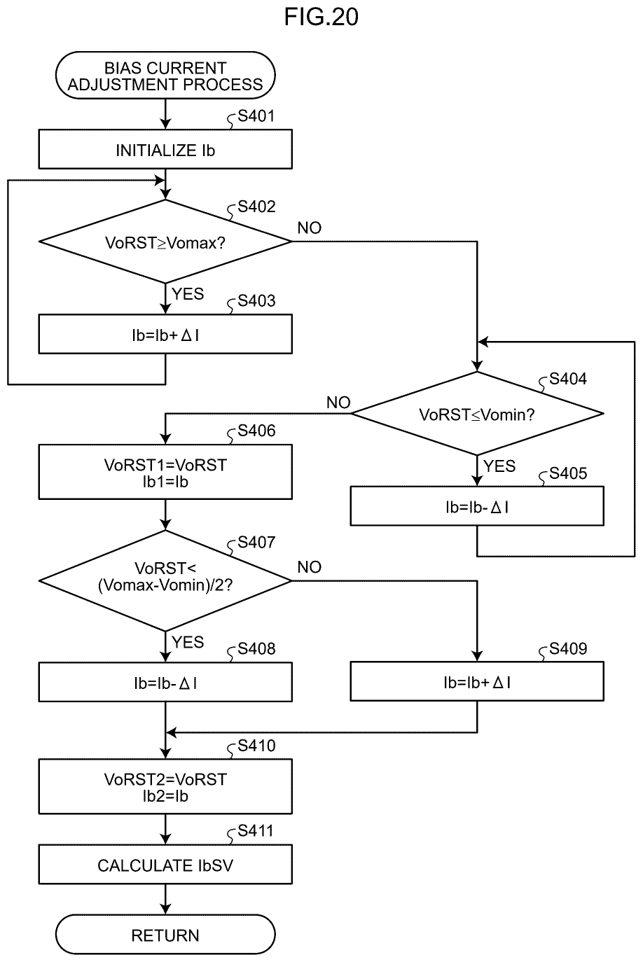

FIG. 20 is a sub-flowchart illustrating an example of the bias current adjustment process. In the bias current adjustment process illustrated in FIG. 20, the control circuit 30 first initializes the bias current Ib of the constant-current source 32 (Step S401). The initial value of the bias current Ib in the bias current adjustment process illustrated in FIG. 20 has been preset.

The control circuit 30 turns on (conducting state) both the reset transistor 81 and the readout transistor 85 to acquire the input potential VoRST of the AFE 31, and determines whether the acquired input potential VoRST of the AFE 31 is equal to or higher than the upper limit input potential Vomax of the AFE 31 (Step S402).

The input potential VoRST acquired in the initial setting process may be, for example, the average value of the input potentials VoRST acquired from the optical sensors WA in the detection area SA, in the same way as in the first embodiment, as illustrated in FIG. 15, or the average value of the input potentials VoRST of the AFE 31 acquired from the optical sensors WA in the segmented areas Block2 and Block3 illustrated in FIG. 7.

Referring back to FIG. 20, if the input potential VoRST of the AFE 31 is equal to or higher than the upper limit input potential Vomax of the AFE 31 (VoRST≥Vomax; Yes at Step S402), the control circuit 30 adds ΔI to the bias current Ib (Ib=Ib+ΔI; Step S403) to obtain the input potential VoRST of the AFE 31, and repeatedly performs the processes at Steps S402 and S403 (first process). ΔI that is added to the bias current Ib at Step S403 is 0.5 μA, for example.

If the input potential VoRST of the AFE 31 is lower than the upper limit input potential Vomax of the AFE 31 (VoRST<Vomax; No at Step S402), the control circuit 30 then determines whether the input potential VoRST of the AFE 31 is equal to or lower than the lower limit input potential Vomin of the AFE 31 (Step S404).

If the input potential VoRST of the AFE 31 is equal to or lower than the lower limit input potential Vomin of the AFE 31 (VoRST≤Vomin; Yes at Step S404), the control circuit 30 subtracts ΔI from the bias current Ib (Ib=Ib−ΔI; Step S405) to obtain the input potential VoRST of the AFE 31, and repeatedly performs the processes at Steps S404 and S405 (second process). ΔI that is subtracted from the bias current Ib at Step S405 is 0.5 μA, for example.

If the input potential VoRST of the AFE 31 exceeds the lower limit input potential Vomin of the AFE 31 (VoRST>Vomin; No at Step S404), the control circuit 30 holds the input potential VoRST of the AFE 31 as a first input potential VoRST1 and the bias current Ib as a first bias current Ib1 (Step S406).

The control circuit 30 then determines whether the input potential VoRST of the AFE 31 is lower than the intermediate potential between the upper limit input potential Vomax and the lower limit input potential Vomin of the AFE 31 (Step S407). The intermediate potential is expressed as (Vomax−Vomin)/2.

If the input potential VoRST of the AFE 31 is lower than the intermediate potential between the upper limit input potential Vomax and the lower limit input potential Vomin of the AFE 31 (VoRST<(Vomax−Vomin)/2; Yes at Step S407), the control circuit 30 subtracts ΔI from the bias current Ib (Ib=Ib−ΔI; Step S408) to obtain the input potential VoRST of the AFE 31, and holds the obtained input potential VoRST of the AFE 31 as a second input potential VoRST2 and the bias current Ib as a second bias current Ib2 (Step S410). If the input potential VoRST of the AFE 31 is equal to or higher than the intermediate potential between the upper limit input potential Vomax and the lower limit input potential Vomin of the AFE 31 (VoRST≥(Vomax−Vomin)/2; No at Step S407), the control circuit 30 adds ΔI to the bias current Ib (Ib=Ib+ΔI; Step S409) to obtain the input potential VoRST of the AFE 31, and holds the obtained input potential VoRST of the AFE 31 as the second input potential VoRST2 and the bias current Ib as the second bias current Ib2 (Step S410) (second process). ΔI that is added to or subtracted from the bias current Ib at Step S408 or S409 is 0.5 μA, for example.

The control circuit 30 then uses Expressions (3) and (4) given below to derive the threshold voltage Vth of the source follower transistor 83 included in the optical sensor WA and a resistance component R of the signal transmission path between the optical sensor WA and the detection circuit 15, and uses Expression (5) given below to calculate a bias current IbSV to be applied in the scan process illustrated in FIG. 9 (Step S411). The resistance component R of the signal transmission path includes the wiring resistance of the signal line 7 and on-resistance of the switches (switches SW1, SW2, SW3, and SW4) included in the multiplexer 40.

$$V Reset - ( V th + R \times Ib 1 ) = V oRST 1 \qquad ( 3 )$$

$$V Reset - ( V th + R \times Ib 2 ) = V oRST 2 \qquad ( 4 )$$

$$V Reset - ( V th + R \times Ib S V ) = V otarget \qquad ( 5 )$$

The input potential target value Votarget of the AFE 31 in Expression (5) given above represents the target value of the input potential VoRST of the AFE 31, in the same way as in the first embodiment. The input potential target value Votarget of the AFE 31 is preset within a target setting range VoTR of 80% to 90% with respect to the upper limit input potential Vomax of the AFE 31 (refer to FIG. 16).

The following describes a specific example of the operation in the bias current adjustment process illustrated in FIG. 20.

As described above, if the input potential VoRST of the AFE 31 is equal to or higher than the upper limit input potential Vomax of the AFE 31 (VoRST≥Vomax; Yes at Step S402), the control circuit 30 adds ΔI to the bias current Ib until the input potential VoRST of the AFE 31 becomes lower than the upper limit input potential Vomax of the AFE 31 (VoRST<Vomax; No at Step S402) (first process).

If the input potential VoRST of the AFE 31 is equal to or lower than the lower limit input potential Vomin of the AFE 31 (VoRST≤Vomin; Yes at Step S404), the control circuit 30 subtracts ΔI from the bias current Ib until the input potential VoRST of the AFE 31 exceeds the lower limit input potential Vomin of the AFE 31 (VoRST>Vomin; No at Step S404) (second process).

If the input potential VoRST of the AFE 31 exceeds the lower limit input potential Vomin of the AFE 31 (VoRST>Vomin; No at Step S404) and the input potential VoRST of the AFE 31 is lower than the upper limit input potential Vomax of the AFE 31 (VoRST<Vomax; No at Step S402), the control circuit 30 uses Expressions (3) to (5) given above to calculate the bias current IbSV to be applied in the scan process illustrated in FIG. 9 (Step S411) (third process).

More specifically, the control circuit 30 uses the input potentials (first input potential VoRST1 and second input potential VoRST2) of the AFE 31 obtained by applying the different bias currents (first bias current Ib1 and second bias current Ib2, respectively) to derive the threshold voltage Vth of the source follower transistor 83 included in the optical sensor WA and the resistance component R of the signal transmission path between the optical sensor WA and the detection circuit 15. The control circuit 30 then sets the bias current Ib as the bias current IbSV such that a value obtained by subtracting the threshold voltage Vth of the source follower transistor 83 and the potential drop corresponding to the resistance component R of the signal transmission path between the optical sensor WA and the detection circuit 15 from the reset potential VReset becomes the input potential target value Votarget of the AFE 31.

Referring back to the initial setting process illustrated in FIG. 19, the control circuit 30 sets the bias current IbSV calculated at Step S411 as a bias current IbSVR when the first light-emitting elements 22R are turned on in the scan process illustrated in FIG. 9 (Step S203a).

The control circuit 30 then turns off the first light-emitting elements 22R (Step S204), turns on the second light-emitting elements 22G (Step S205), and performs the bias current adjustment process to set a bias current IbSVG to be applied when the second light-emitting elements 22G are turned on in the scan process illustrated in FIG. 9 (Step S400). The bias current adjustment process to set the bias current IbSVG is the same as the above-described bias current adjustment process to set the bias current IbSVR when the first light-emitting elements 22R are turned on, and therefore, will not be described in detail.

Referring back to the initial setting process illustrated in FIG. 19, the control circuit 30 sets the bias current IbSV calculated at Step S411 as the bias current IbSVG when the second light-emitting elements 22G are turned on in the scan process illustrated in FIG. 9 (Step S206a).

The control circuit 30 then turns off the second light-emitting elements 22G (Step S207), turns on the third light-emitting elements 22B (Step S208), and performs the bias current adjustment process to set a bias current IbSVB to be applied when the third light-emitting elements 22B are turned on in the scan process illustrated in FIG. 9 (Step S400). The bias current adjustment process to set the bias current IbSVB is the same as the bias current adjustment process to set the bias current IbSVR when the first light-emitting elements 22R are turned on and the bias current adjustment process to set the bias current IbSVG when the second light-emitting elements 22G are turned on, and therefore, will not be described in detail.

Returning to the initial setup process shown in FIG. 19, the control circuit 30 sets the bias current IbSV calculated in step S411 as the bias current IbSVB when the third light-emitting elements 22B are turned on in the scan process shown in FIG. 11 (step S209a).

The control circuit 30 then turns off the third light-emitting elements 22B (Step S210), and ends the initial setting process illustrated in FIG. 19.

When performing the scan process illustrated in FIG. 9, the detection device 1 according to the second embodiment applies the reset potential set value VResetSV and the reference potential set value VCOMSV that have been set after the initial setting process illustrated in FIG. 19. As a result, the changes over time of the state of the samples to be cultured 100 can fall within the detection range in the detection circuit 15 regardless of sensor-panel-to-sensor-panel variation in the threshold voltage Vth of each of the transistors included in the optical sensor WA.

As described above, in the initial setting process before starting the scan process, the detection device 1 according to the second embodiment adjusts the bias current Ib to flow through the signal transmission path between the optical sensor WA and the detection circuit 15, and when performing the subsequent scan process, the detection device 1 applies the bias current IbSV set in the initial setting process. As a result, the changes over time of the state of the samples to be cultured 100 can be acquired properly regardless of sensor-panel-to-sensor-panel variation in the threshold voltage Vth of each of the transistors included in the optical sensor WA, in the same way as in the first embodiment.

While the preferred embodiments of the present invention has been described above, the present invention is not limited to such embodiments. The content disclosed in the embodiments is merely an example, and can be variously modified within the scope not departing from the gist of the present invention. Any modifications appropriately made within the scope not departing from the gist of the present invention also naturally belong to the technical scope of the present invention. At least one of various omissions, substitutions, and changes of the components can be made without departing from the gist of the embodiments described above and the modifications thereof. For example, the detection device may be configured to, in the initial setting process, adjust a set value of the reset potential, a set value of the bias current, or both.

Other operational advantages accruing from the aspects described in the embodiments herein that are obvious from the description herein, or that are conceivable as appropriate by those skilled in the art will naturally be understood as accruing from the present disclosure.

What is claimed is:

1. A detection device comprising:
a plurality of optical sensors each comprising:
a photodiode;
a reset transistor configured to apply a reset potential to a cathode of the photodiode;
a source follower transistor configured to output a signal corresponding to a potential generated by the photodiode; and
a readout transistor configured to read out an output signal of the source follower transistor;
a sensor panel having a detection area in which the optical sensors are arranged in a planar configuration;
a light source configured to emit light to an object to be detected placed between the light source and the sensor panel;
a detection circuit configured to acquire a potential corresponding to a bias current that flows through the readout transistor; and
a control circuit configured to control the sensor panel, the light source, and the detection circuit, wherein
the control circuit is configured to perform an initial setting process to adjust at least one of a set value of the reset potential or a set value of the bias current such that an input potential acquired by the detection circuit via a signal transmission path between each of the optical sensors and the detection circuit falls within a predetermined target setting range by controlling the reset transistor and the readout transistor to be on while the object to be detected is not placed between the sensor panel and the light source.

2. The detection device according to claim 1, wherein the target setting range is a range from 80% to 90% with respect to an upper limit input value of the detection circuit.

3. The detection device according to claim 1, wherein the control circuit is configured to adjust the set value of the reset potential and a set value of a reference potential to be applied to an anode of the photodiode such that a potential difference between ends of the photodiode is constant, in the initial setting process.

4. The detection device according to claim 1, wherein the control circuit is configured to adjust at least one of the set value of the reset potential or the set value of the bias current such that an average of a plurality of the input potentials corresponding to the optical sensors falls within the predetermined target setting range, in the initial setting process.

5. The detection device according to claim 1, wherein the control circuit is configured to adjust at least one of the set value of the reset potential or the set value of the bias current such that an average of a plurality of the input potentials corresponding to some of the optical sensors included in the detection area falls within the predetermined target setting range, in the initial setting process.

6. The detection device according to claim 1, wherein
the optical sensors are arranged in a matrix having a row-column configuration along a first direction and a second direction intersecting the first direction in the detection area of the sensor panel, and
the control circuit is configured to adjust at least one of the set value of the reset potential or the set value of the bias current such that, among the optical sensors, an average of a plurality of the input potentials corresponding to multiple optical sensors that are included in the detection area and are apart from one another by predetermined distances in the first direction and the second direction falls within the predetermined target setting range, in the initial setting process.

7. The detection device according to claim 1, wherein
the detection area of the sensor panel is divided into a plurality of segmented areas, and
the control circuit is configured to adjust at least one of the set value of the reset potential or the set value of the bias current such that, among the optical sensors, an average of a plurality of the input potentials corresponding to multiple optical sensors included in some of the segmented areas falls within the predetermined target setting range, in the initial setting process.

8. The detection device according to claim 1, wherein
the light source is configured to emit light in a plurality of colors different from one another to the object to be detected in a time-division manner, and
the control circuit is configured to adjust at least one of the set value of the reset potential or the set value of the bias current for each emission color of the light source, in the initial setting process.

9. The detection device according to claim 1, wherein
the initial setting process comprises:
a first process to reduce the set value of the reset potential until the input potential becomes lower than an upper limit input value of the detection circuit;
a second process to increase the set value of the reset potential until the input potential exceeds a lower limit input value of the detection circuit; and
a third process to calculate the set value of the reset potential by adding a difference between a predetermined value within the predetermined target setting range and the input potential to the reset potential, when the input potential exceeds the lower limit input value and is lower than the upper limit input value.

10. The detection device according to claim 1, wherein
the initial setting process comprises:
a first process to reduce the set value of the bias current until the input potential becomes lower than an upper limit input value of the detection circuit;
a second process to increase the set value of the bias current until the input potential exceeds a lower limit input value of the detection circuit; and
a third process to calculate the set value of the bias current such that a value obtained by subtracting a threshold voltage of the source follower transistor and a potential drop corresponding to a resistance component of the signal transmission path from the reset potential becomes a predetermined value within the predetermined target setting range, when the input potential exceeds the lower limit input value and is lower than the upper limit input value.
